(19) United States
(12) Patent Application Publication
(10) Pub. No.: US 2006/0025728 A1
(43) Pub. Date: Feb. 2, 2006

(54) Device and method for colonic lavage

(76) Inventors: Arnold R. Leiboff, Stony Brook, NY (US); Jay Leiboff, Fair Lawn, NJ (US); Joseph Zipper, Miller Place, NY (US)

Correspondence Address: BRIAN ROFFE, ESQ, 11 SUNRISE PLAZA, SUITE 303, VALLEY STREAM, NY, 11580-6170, US

(21) Appl. No.: 11/189,305

(22) Filed: Jul. 26, 2005

Related U.S. Application Data
(60) Provisional application No. 60/592,837, filed on Jul. 30, 2004.

(51) Int. Cl. A61M 1/00

(52) U.S. Cl. 604/317

(57) ABSTRACT

Colonic lavage device including a tubular body having a forward portion with an opening at a front end and a rearward portion including an integral seal defining an opening. A side tube includes an outlet for fecal matter discharge thereby defining a passage from the opening at the front end of the forward portion to the outlet. An integral nipple or end cap closes the seal opening. The forward portion is inserted into a patient's bowel and an irrigating tube is passed through the seal, once the nipple or end cap is cut off, to a position beyond the front end of the forward portion and in the bowel. Irrigating fluid is passed through the irrigating tube to cleanse the bowel and fecal matter from the bowel flows through the tubular body and is discharged therefrom through the side tube outlet, e.g, to a discharge tube leading to a container.

### CROSS-REFERENCE TO RELATED APPLICATION

This application claims priority under 35 U.S.C. §119(e) of U.S. provisional patent application Ser. No. 60/592,837 filed Jul. 30, 2004, which is incorporated by reference herein.

### FIELD OF THE INVENTION

The present invention relates generally to a device for colonic lavage, and to a method and system for using such a device, and in particular for intra-operative on-table lavage.

### BACKGROUND OF THE INVENTION

In emergency surgery, a clean, well-prepared bowel is required for safe colonic resection and anastomosis. Various colonic lavage devices have been developed to irrigate or clean the bowel.

In U.S. Pat. No. 4,637,814, incorporated by reference herein, the inventor presented several innovative apparatus and methods for irrigating and cleaning a colon in preparation for subsequent colonic surgery. One apparatus described in this patent includes a drain tube having a tubular body and a single side arm or side tube. In use, a proximal end of the tubular body is inserted into the colon and an irrigation tube is inserted into a distal end of the tubular body and pushed forward to extend beyond the proximal end of the tubular body and into the colon. Irrigating fluid is directed through the irrigation tube to clean fecal matter from the colon. Effluent generated during the colonic irrigation procedure flows into the tubular body and the side tube and is removed through a tube connected to the side tube. Effluent is prevented from passing through the distal end of the tubular body by a bushing fixed to the tubular body which cooperates with a cuff member surrounding the irrigation tube to thereby seal the space between the irrigating tube and the distal end of the tubular body. Additional details about this apparatus, related apparatus and methods for using the same can be found in the '814 patent and in Intraoperative High-flow Antegrade Irrigation by Arnold R. Leiboff et al., Diseases of the Colon & Rectum, May 1985, Vol. 28, No. 5, pages 323-332.

A similar drain tube having a tubular body and a single side tube is described in U.S. Pat. No. 6,761,702 (Smith/Intermark Medical Innovation Ltd.). The drain tube described in this patent has a port at the distal end of the tubular body and like the '814 patent, incorporates a sealing mechanism designed to prevent leakage of effluent from the tubular body. The sealing mechanism includes an annular seal bonded to the tubular body.

U.S. Pat. No. 5,443,445 (Peters et al./Clinical Product Development Limited) describes an intra-operative colon irrigation system which includes a device having a tubular body with a forward end formed by a dome-shaped nozzle to prevent intussusception of the bowel, and a side tube including an outlet for the discharge of fecal matter from the device. A port is located at the other end of the tubular body, for the insertion of an ultrasonic device or for the injection of water or air under pressure.

Hepworth et al., in Gut 1999; 44 (suppl.1), A134: TH533, have proposed a retrograde procedure for on-table lavage which comprises advancing a jet of irrigation fluid through the bowel as the lavage process proceeds.

A known device for the lavage method of Hepworth et al. comprises a rigid Y-connector to which three flexible tubes are connected. One tube is inserted in the bowel and another carries irrigation fluid, while the third leads to a container for the irrigated fecal material. This device has not proved ideal. In particular, the rigid Y-tube easily becomes blocked with fecal material and it is difficult to advance the irrigation fluid tube through the Y-connector as the lavage process proceeds. Furthermore, the “Y” configuration of the connector prevents the insertion of a colonoscope through the device, a colonoscope being used after lavage to check the condition of the bowel.

### OBJECTS AND SUMMARY OF THE INVENTION

It is an object of the present invention to provide new and improved colonic lavage devices and methods.

It is another object of the present invention to provide new and improved systems and methods for colonic lavage using a new and improved colonic lavage device.

It is yet another object of the present invention to provide a new method for manufacturing a colonic lavage device.

In order to achieve these objects and others, a device for colonic lavage in accordance with the invention includes a tubular body including a forward portion having an opening at a front end and a rearward portion including an integral seal defining an opening. A side tube is connected to the tubular body and has an outlet at a rear end. A continuous passage is defined between the opening at the front end of the forward portion of the tubular body and the outlet of the side tube. The forward portion of the tubular body is designed for insertion into and connection to the bowel of the patient so that an irrigating tube can be passed through the integral seal to a position beyond the front end of the forward portion and in the patient's bowel. Irrigating fluid is then passed through the irrigating tube to cleanse the bowel and fecal matter from the bowel flows in the passage through the tubular body and the side tube and is discharged from the device through the outlet of the side tube, e.g., to a discharge tube leading to a container.

By forming the tubular body with an integral seal, a separate sealing mechanism is not required as in prior art constructions of colonic lavage devices. There is therefore no issue of bonding or fixing such a separate sealing mechanism to the tubular body, which adds a manufacturing step to the formation of the device.

The seal is preferably circular since the irrigating tube usually has a circular cross-sectional shape. Nevertheless, for use with non-circular irrigating tubes, the seal can be constructed to define other shapes of openings.

To facilitate the introduction of the irrigating tube into the bowel through the device, the opening defined by the seal is preferably arranged in a straight line relationship with the opening at the front end of the forward portion of the tubular body. Instead of an irrigating tube, other instruments can also be passed through the opening defined by the seal to a position beyond the front end of the forward portion of the tubular body, e.g., a colonoscope.

The tubular body and side tube are preferably formed integral with one another so that the entire device has a one-piece unitary form.

To prevent fecal matter from leaking out of the colonic lavage device when it is connected to the colon and before the surgeon is ready to irrigate the colon by inserting the irrigating tube through the seal, the opening defined by the seal must be closed.

To close the opening defined by the seal, one embodiment of the device includes an integral nipple projecting rearward from the seal. The integral nipple must be cut off once the surgeon is ready to irrigate the bowel in order to enable the irrigating tube to be passed into and through the device. In one embodiment, the seal and nipple have a wall thickness substantially smaller than the wall thickness of the forward portion of the tubular body so that the nipple is easily severable by a standard surgical suture scissors and so that the seal is easily stretchable to accommodate the passage of and to seal against the surface of tubular devices of varying diameters.

In another embodiment, to close the opening defined by the seal, the device includes an integral end cap arranged rearward of the seal and in connection with the rearward portion of the tubular body. Like the nipple, the integral end cap must be cut off in order to enable the irrigating tube to be passed into and through the device.

The forward portion of the tubular body may include corrugations and a sewing cuff or ring arranged rearward thereof to facilitate securing the colon to the tubular body. The sewing ring enables the colon to be held in position while it is tied or clamped around the corrugations of the tubular body.

A system for colonic lavage using the above-described colonic lavage device would include a source of irrigating fluid, the irrigating tube which passes irrigating fluid from the irrigating fluid source into the colon, a sealable container for effluent from the colonic lavage and a discharge tube connected at one end to the outlet of the side tube and at an opposite end to the container. The irrigating tube is coupled at one end to the irrigating fluid source and the opposite end passes through the tubular body to extend beyond the front end of the forward portion into the colon. The irrigating tube passes through the opening defined by the seal and sealingly engages the seal. Effluent from the colon, i.e., irrigating fluid with fecal matter, flows through the passage in the colonic lavage device into the discharge tube and through the discharge tube into the container.

A method for colonic lavage using the above-described colonic lavage device involves inserting the forward portion of the tubular body into the colon of a patient, securing the colon to the tubular body, cutting off the integral nipple or end cap, inserting an irrigating tube through the opening defined by the seal, through the tubular body and through the opening at the front end of the forward portion of the tubular body to a position in the colon, passing irrigation fluid through the irrigating tube to cleanse fecal matter from the colon and discharging fecal matter through the passage and out of the outlet in the side tube. The irrigating tube may be advanced through the device into the colon as desired as fecal matter is cleansed from the colon. Securing the colon to the tubular body may entail first clamping the colon to a sewing ring formed on the forward portion of the colonic lavage device, sewing the edge of the colon to the sewing ring and further securing the colon with ties around the colon and the corrugations formed on the forward portion of the device.

### BRIEF DESCRIPTION OF THE DRAWINGS

The invention, together with further objects and advantages thereof, may best be understood by reference to the following description taken in conjunction with the accompanying drawings, wherein like reference numerals identify like elements, and wherein:

FIG. 1 is a longitudinal cross-sectional view of a first embodiment of a colonic lavage device according to the invention;

FIG. 2 is a longitudinal cross-sectional view of a second embodiment of a colonic lavage device according to the invention shown;

FIG. 3 is a longitudinal cross-sectional view of the colonic lavage device shown in FIG. 1, shown with a sewing ring and shown when used in a colonic lavage system; and

FIG. 4 is a front view of a mold for manufacturing the colonic lavage device shown in FIG. 1.

### DETAILED DESCRIPTION OF THE INVENTION

Referring to the accompanying drawings, a first embodiment of a colonic lavage device in accordance with the invention is designated generally as 10 and comprises an integral tubular body 12 having a length of about 180 mm and having a corrugated, elongate forward portion 14 and a rearward portion 16. An unobstructed circular opening 18 is formed at the front end of the forward portion 14. The tubular body 12 may be formed of a medical grade plastic material and with an appropriate thickness as known to those skilled in the art, e.g., from PVC with thickness of about 2 mm to about 3 mm.

The device 10 also includes a side tube 20 connected at a front end to the tubular body 12 and oriented at an angle to the forward portion 14. Side tube 20 has a substantially circular outlet 22 at a rear end such that a continuous passage 24 is defined from the opening 18 to the outlet 22, i.e., through the interior of the tubular body 12 and through the interior of the side tube 20. The rear end portion of the side tube 20 may be narrowed as shown. Although not shown, the side tube 20 may be shaped to constitute a hand grip to ease control of the device 10 by a surgeon.

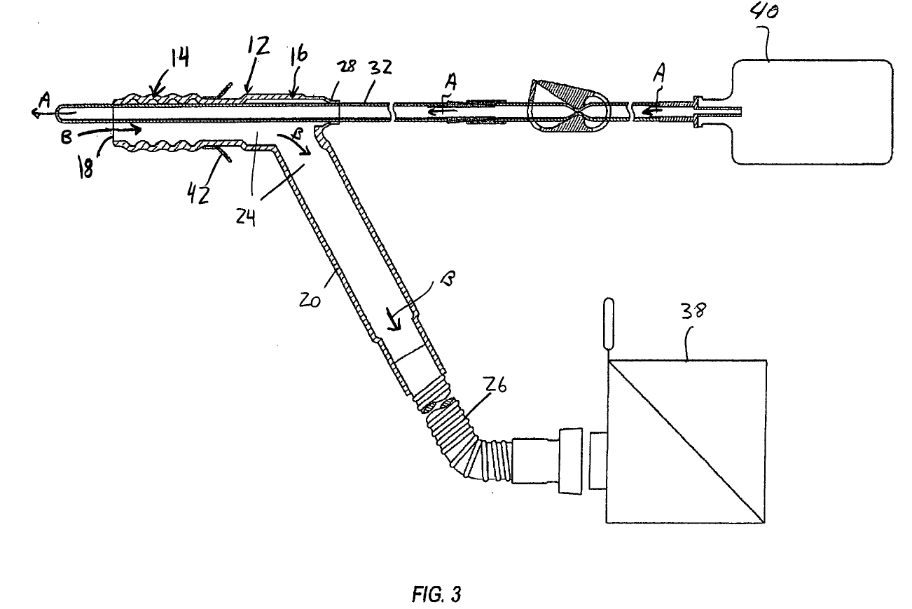

A discharge tube 26 is connected to the side tube 20, e.g., sealed thereto, and extends from the outlet 22 thereof to a sealable effluent container 38 (see FIG. 3). Discharge tube 26 may be connected to the container 38 in any manner known in the art for connecting tubes to containers, e.g., a threaded connection, a snap-fit connection, a press-fit connection and the like.

The rearward portion 16 of the tubular body 12 includes an integral seal 28 defining a circular opening 30 through which an irrigating tube 32 is operatively passed (see FIG. 3). The term “integral” is being used herein to mean that the seal 28 is formed as a one-piece construction with and as part of the rearward portion 16 of the tubular body 12.

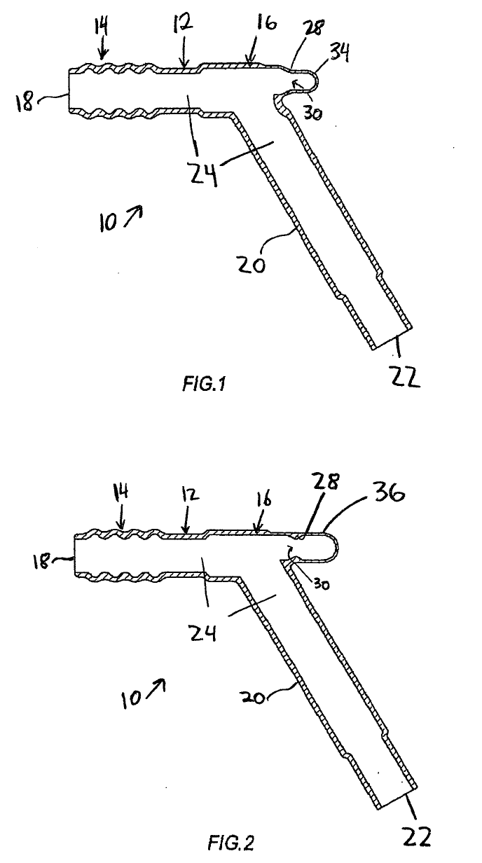

In the embodiment shown in FIG. 1, to close the opening 30 in order to prevent leakage of fecal matter from the colon before the surgeon is ready to perform the bowel irrigation via the irrigating tube 32, an integral nipple 34 projects rearward from the seal 28 around the opening 30. The integral nipple 34 must be cut off when the surgeon is ready to irrigate the bowel in order to enable the irrigating tube 32 to be passed into and through the tubular body 12.

Since the integral seal 28 extends inward from the rear edge of the rearward portion 16, the diameter of the opening 30 defined by the integral seal 28 is less than the diameter of a surrounding part of the rearward portion 16. The diameter of the opening 30 should be substantially the same as, or slightly smaller than, the diameter of the irrigating tube 32 (at that portion which will engage with the integral seal 28) to provide a tight seal and thereby prevent effluent from being discharged out of the rearward portion 16 of the tubular body 12. Alternatively, if a cuff member is used in connection with the irrigating tube 32 (as in one embodiment disclosed in U.S. Pat. No. 4,637,814 discussed above), then the diameter of the integral seal 28 may be larger than the diameter of the irrigating tube 32 in which case, the cuff member surrounding the irrigating tube 32 will sealingly engage with the integral seal 28.

The opening 30 defined by the integral seal 28 is preferably in straight line relationship with the opening 18 at the front end of the forward portion 14 of the tubular body 12. This enables the irrigating tube 32 to be more easily advanced through the device 10 during use, without any bends or turns, and also enables alternative components, such as a colonoscope, to be inserted through the device 10 into the colon of the patient.

In the embodiment shown in FIG. 2, to prevent leakage of fecal matter from the colon through the opening 30 before the surgeon is ready to perform the bowel irrigation via the irrigating tube 32, the opening 30 is closed by an integral end cap 36 connected to the outer periphery of the rearward portion 16 and arranged rearward of the integral seal 28. In view of the formation of the tubular body 12 with the integral end cap 36, there is no conduit through the rearward portion 16 (through which fecal matter might inadvertently leak) until the end cap 36 is operatively cut off and removed. Cutting off the end cap 36 causes the integral seal 28 to be exposed, at which time, the irrigating tube 32 is then passed through the integral seal 28 to conduct the irrigation of the bowel.

The formation of the tubular body 12 with the integral seal 28 and the integral nipple 34 or end cap 36 thus differs from prior art constructions of tubular bodies for similar colonic lavage devices, such as disclosed in U.S. Pat. Nos. 4,637,814 and 6,761,702, wherein the distal end of the tubular body is sealed by one or more members which are not integrally formed with the tubular body. Rather, the seals are formed as separate components which must be fixed or bonded to the rearward portion of the tubular body. For example, in U.S. Pat. No. 6,761,702, an annular seal is formed as a separate component from the tubular body and is therefore required to be bonded thereto by an adhesive. Forming a seal integral with the tubular body as in the invention therefore eliminates the need to manufacture additional components, the need for bonding an additional component to the tubular body and associated risks of leakage resulting from potentially deficient bonding of the components.

Nevertheless, it is not necessary to manufacture the device 10 with an integral nipple 34 or end cap 36, or another integral mechanism for closing the opening 30 defined by the integral seal 28, but rather, the device 10 can be manufactured and/or sold with a non-integral closing mechanism or with an open rearward portion 16 with other means to prevent leakage of fecal matter through the opening 30 defined by the integral seal 28 prior to irrigation. In this case, the integral seal 28 may be formed at any location in the rearward portion, e.g., inward from or at the rear edge of the rearward portion 16 of the device 10.

One example of a non-integral mechanism for closing the opening 30 defined by the integral seal 28 is an end cap, separately formed from the tubular body 12 and bonded or otherwise attached to the rearward portion 16 of the tubular body 12.

The tubular body 12 also optionally includes a circumferential sewing cuff or sewing ring 42 arranged rearward of the corrugations of the forward portion 14 (see FIG. 3). The sewing ring 42 is designed to enable the edge of the bowel to be easily and securely clamped against it using surgical clamps after which the edge of the bowel is sewed thereto so that the bowel can be securely held to the tubular body 12 while and after ties are wrapped around the colon and corrugations. The sewing ring 42 may be made of a soft, elastic, pliable plastic separate from the tubular body 12 or integral therewith. Additional details about the sewing ring 42 are provided in the inventor's U.S. patent application Ser. No. 10/672,053, the specification of which is hereby incorporated by reference herein in its entirety.

The device 10 can be manufactured by a dip molding, rotational molding or blow molding process, or other similar process. During the manufacturing process, the seal 28, nipple 34 and/or end cap 36 can be formed partially or entirely with a substantially smaller thickness than the remaining portion of the tubular body 12 to facilitate cutting thereof by the surgeon using a standard surgical suture scissors.

Furthermore, the device 10 may be formed from a flexible, transparent material to enable the surgeon to view the flow of effluent through the passage 24 and identify and dislodge any blockage which may occur.

The irrigating tube 32 should have a length exceeding the length of the tubular body 12, and should be considerably longer to enable the irrigating tube 32 to be advanced as required through the colon of the patient, while continuing to be connected to the irrigation fluid source. To assist the advance of the irrigating tube 32 through the integral seal 28, the irrigating tube 32 and/or integral seal 28 might be formed from a low-friction material or coated with such a material.

The irrigating tube 32 may be any known irrigating tube used to pass a fluid through. Preferably, the irrigating tube 32 includes an aperture at a tip as shown. A rounded tip may be molded from and integral with the irrigating tube 32, rather than being a separately formed “nozzle” bonded to the tube, as described in U.S. Pat. No. 6,761,702.

In use for colonic lavage, the colonic lavage device 10 is connected to the discharge tube 26 by connecting the discharge tube 26 to the outlet end of the side tube 20. Discharge tube 26 is connected at its opposite end to the sealable container 38, the sealable container 38 possibly being the type of container disclosed in U.S. Pat. No. 6,761,702.

The forward portion 14 of the tubular body 12 is then inserted into the proximal bowel of a patient, e.g. following resection of a tumor. The sewing ring 42 and the corrugated construction of the forward portion 14 of the tubular body 12 enable the bowel to be secured thereto in a leak-free manner. Preferably the edge of the open end of bowel is clamped to the sewing ring 42 by clamps and then sutured to the sewing ring 42. Interrupted sutures can be used at points around the circumference of the bowel, or a continuous circumferential suture may be used. Ties or straps (e.g., cable ties, umbilical tape or silk ties) are then placed around the bowel and forward portion 14 of the tubular body 12 where the corrugations prevent slippage of the ties or straps. By suturing the bowel edge to the sewing ring 42, a secure attachment of the bowel to the tubular body 12 is obtained whereby the tubular body 12 cannot disjoin from the bowel. If the tubular body 12 were to slip out of the bowel, the sterile operative field could become contaminated, increasing the likelihood of postoperative infection. Another advantage of the placement of a sewing ring 42 on the tubular body 12 is that it allows for rapid and secure fixation of the bowel edge to the tubular body 12 with surgical clamps when the tubular body 12 is first introduced into the bowel. This allows the surgeon to hold the inserted tubular body 12 in the bowel securely, while sewing the bowel edge to the sewing ring 42 and tying the bowel around the tubular body 12.

At this time, fecal matter drains from the colon into the passage 24 in the device 10 and flows through the passage 24 out of the outlet 22 of the side tube 20, into the discharge tube 26 and to the container 38. However, fecal matter cannot leak out through the opening 30 in the seal 28 in view of the presence of the nipple 34 or end cap 36, which have not been cut off yet.

Once the device 10 is in place with the colon secured thereto and the surgeon is ready to begin irrigating the colon, the nipple 34 or end cap 36 is then cut off, e.g., using surgical scissors, to expose the integral seal 28, and the irrigating tube 32 is then passed through the exposed integral seal 28 to extend through the tubular body 12 and the opening 18 into the colon of the patient.

Once the irrigating tube 32 is in the position shown in FIG. 3, irrigating fluid is passed from a source thereof 40 through the irrigating tube 32 and out of the irrigating tube 32 through a tip thereof as shown by the arrows A, to cleanse obstructed fecal matter from the colon. The irrigating tube 32 may be advanced by hand through the device further into the colon as fecal matter is cleansed therefrom. Although not shown in the drawings, a clamp or other controllable closing device may be provided at a convenient position along the length of the irrigating tube 32, upstream of the device 10, to enable the operator to control the rate of flow of irrigating fluid into the colon.

The irrigation fluid is preferably a liquid, although the device is suitable for use with compressed air, or a mixture of air and liquid. A suitable liquid is a saline solution contained in a saline bag.

Effluent, i.e., irrigation fluid and fecal matter, returns to the tubular body 12 to be discharged through the outlet 22 of the side tube 20, as shown by the arrows B, and into the discharge tube 26 (see FIG. 3). Discharge tube 26 leads to the sealable container 38. The discharge tube 26 is flexible and preferably has a clear construction to enable the surgeon to see when the effluent is running clean, indicating that the lavage process is complete. Appropriate dimensioning of the integral seal 28 ensures that the irrigating tube 32 is in sealing engagement with the integral seal 28 to thereby prevent flow of effluent through the distal end of the tubular body 12.

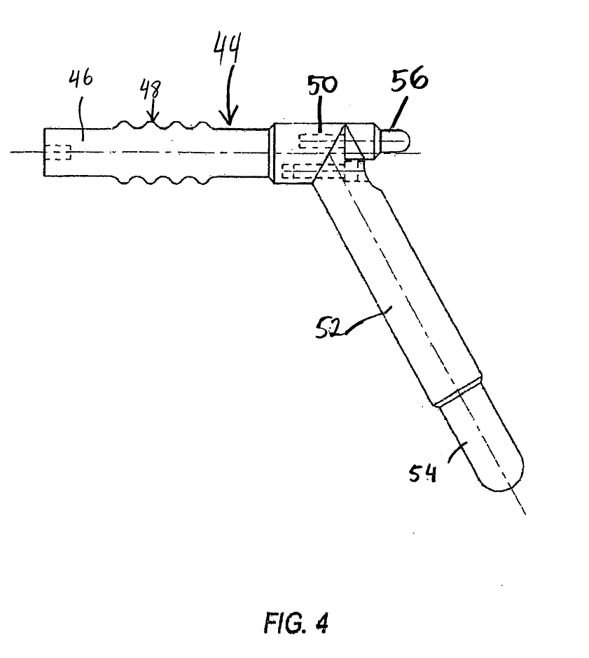

Referring now to FIG. 4, a method for manufacturing the device 10 (the embodiment shown in FIG. 1) will now be explained. The device 10 may be manufactured using a dip molding technique in which a solid mold 44 is inserted into a fluid plastic precursor which covers the mold 44 and a form having the shape of the device 10 is removed from the mold 44. Alternatively, the mold 44 can be designed to provide a form which can readily be worked to obtain the shape of the device 10, e.g., the side tube 20 can be formed with a closed end which is then simply cut off and removed.

To this end, the mold 44 includes a first substantially cylindrical portion 46 which will form the forward portion 14 of the tubular body 12 and therefore includes an undulating surface portion 48 which will form the corrugations in the forward portion 14, a rear portion 50 which will form the rearward portion 16 with the integral seal 28 and nipple 34 and substantially cylindrical portions 52, 54 at an angle to the rear portion 50 which will form the side tube 20. The rear portion 50 therefore includes an extension 56 with reduced diameter which will form the seal 28 and nipple 34.

The mold 44 may be formed as a one-piece, homogenous member from an appropriate mold-forming material as known to those skilled in the plastics manufacturing art. However, one problem with such a homogenous mold is that the thickness of the device 10 produced by the mold will be substantially the same throughout the device 10. A problem can therefore arise during use of such a device in that the nipple 34 might be excessively thick and thus not easily cut off by a standard surgical suture scissors or other cutting implements in the operating room.

One solution to this problem is to form the extension 56 of the mold 44 from a material having a lower thermal conductivity than remaining portions of the mold 44. The mold 44 would thus have two parts connected together, e.g., by a screw or threaded engagement, with each part being made of a different material. In this manner, less plastic will solidify around the extension 56 during the dip molding process resulting in a thinner, more easily severable nipple 34. Alternatively, the nipple 34 might simply be shaved to reduce its thickness, although this would involve an additional manufacturing step.

Other methods for manufacturing the device 10 are also contemplated to be within the scope and spirit of the invention. Also, the same manufacturing method described above can be used to form the device 10 shown in FIG. 2, with a different mold of course.

While particular embodiments of the invention have been shown and described, it will be obvious to those skilled in the art that changes and modifications may be made without departing from the invention in its broader aspects, and, therefore, the aim in the appended claims is to cover all such changes and modifications as fall within the true spirit and scope of the invention. For example, although the device 10 is described in conjunction with use for colonic lavage, it is conceivable that the same device has other uses in the medical field and it is intended that the appended claims cover such uses.

1. A device for colonic lavage, comprising
a tubular body including a forward portion having an opening at a front end and a rearward portion including an integral seal defining an opening; and
a side tube including an outlet for the discharge of fecal matter from the device, thereby defining a continuous passage from said opening at said front end of said forward portion of said tubular body to said outlet.

2. The device of claim 1, wherein said seal is arranged at a rear edge of said rearward portion.

3. The device of claim 2, further comprising an integral nipple projecting rearward from said seal to close said opening defined by said seal.

4. The device of claim 3, wherein said seal and said nipple have a wall thickness substantially smaller than the wall thickness of said forward portion of said tubular body, so that said nipple is easily severable by a standard surgical suture scissors and so that said seal is easily stretchable to accommodate the passage of and to seal against the surface of tubular devices of varying diameters.

5. The device of claim 1, further comprising an integral end cap arranged rearward of said seal and in connection with said rearward portion, said end cap closing said opening defined by said seal.

6. The device of claim 5, wherein said end cap has a wall thickness substantially smaller than the wall thickness of said forward portion of said tubular body, so that said end cap is easily severable by a standard surgical suture scissors.

7. The device of claim 1, further comprising a sewing ring arranged on said forward portion for securing said forward portion and an edge portion of severed bowel together.

8. The device of claim 1, wherein said seal is circular such that said opening defined by said seal is substantially circular.

9. The device of claim 1, wherein said seal projects from an inner surface of said rearward portion.

10. The device of claim 1, wherein said opening defined by said seal is arranged in a straight line relationship with said opening at said front end of said forward portion.

11. The device of claim 1, wherein said tubular body and said side tube are integral with one another.

12. A system for colonic lavage, comprising:
a colonic lavage device adapted to be secured to the colon and including a tubular body having a forward portion with an opening at a front end and a rearward portion including an integral seal defining an opening, and a side tube including an outlet for the discharge of fecal matter from the device thereby defining a continuous passage from said opening at said front end of said forward portion of said tubular body to said outlet; a source of irrigating fluid;
an irrigating tube for passing irrigating fluid into the colon, said irrigating tube being coupled at one end to said irrigating fluid source and adapted to pass through said tubular body to a position beyond said front end of said forward portion and in the colon, said irrigating tube passing through said opening defined by said seal and sealingly engaging with said seal;
a sealable container for effluent from the colonic lavage; and
a discharge tube connected at one end to said outlet of said side tube and at an opposite end to said container whereby effluent from the colon flows through said passage into said discharge tube and through said discharge tube into said container.

13. The system of claim 12, wherein said seal is arranged at a rear edge of said rearward portion.

14. The system of claim 13, wherein said colonic lavage device further comprises an integral nipple projecting rearward from said seal to close said opening defined by said seal.

15. The system of claim 14, wherein said seal and said nipple have a wall thickness substantially smaller than the wall thickness of said forward portion of said tubular body, so that said nipple is easily severable by a standard surgical suture scissors and so that said seal is easily stretchable to accommodate the passage of and to seal against the surface of tubular devices of varying diameters.

16. The system of claim 12, wherein said colonic lavage device further comprises an integral end cap arranged rearward of said seal and in connection with said rearward portion, said end cap closing said opening defined by said seal.

17. The system of claim 16, wherein said end cap has a wall thickness substantially smaller than the wall thickness of said forward portion of said tubular body, so that said end cap is easily severable by a standard surgical suture scissors.

18. The system of claim 12, wherein said colonic lavage device further comprises a sewing ring arranged on said forward portion for securing said forward portion and an edge portion of severed bowel together.

19. The system of claim 12, wherein said seal is circular such that said opening defined by said seal is substantially circular.

20. The system of claim 12, wherein said seal projects from an inner surface of said rearward portion.

21. The system of claim 12, wherein said opening defined by said seal is arranged in a straight line relationship with said opening at said front end of said forward portion.

22. The system of claim 12, wherein said tubular body and said side tube are integral with one another.

23. A method for colonic lavage using a device comprising a tubular body including a forward portion having an opening at a front end and a rearward portion including an integral seal defining an opening, and a side tube including an outlet for the discharge of fecal matter from the device thereby defining a continuous passage from the opening at the front end of the forward portion of the tubular body to the outlet, the method comprising:
inserting the forward portion of the tubular body into the colon of a patient,
securing the colon to the tubular body;
inserting an irrigating tube through the opening defined by the seal, through the tubular body and through the opening at the front end of the forward portion of the tubular body to a position in the colon;
passing irrigation fluid through the irrigating tube to cleanse fecal matter from the colon; and
discharging fecal matter through the passage and out of the outlet in the side tube.

24. The method of claim 23, wherein the device further includes an integral nipple projection rearward from the seal, further comprising cutting off the nipple prior to inserting the irrigating tube through the opening.

25. The method of claim 23, wherein the device further includes an integral end cap arranged rearward of the seal and in connection with said rearward portion, further comprising cutting off the end cap prior to inserting the irrigating tube through the opening.

26. The method of claim 23, wherein the device further includes a sewing ring arranged on the forward portion, the step of securing the colon to the tubular body comprises clamping an edge portion of the colon to the sewing ring, sewing the edge of the colon to the sewing ring and placing ties around the colon and forward portion of the tubular body.
